(19) United States
(12) Patent Application Publication
(10) Pub. No.: US 2022/0202128 A1
(43) Pub. Date: Jun. 30, 2022

(54) SAFETY HELMET WITH DISPOSABLE SANITARY FACE SHIELD AND METHOD

(71) Applicant: ERB Industries, Inc., Woodstock, GA (US)

(72) Inventor: Christopher T. Padgett, Jasper, GA (US)

(21) Appl. No.: 17/566,789

(22) Filed: Dec. 31, 2021

Related U.S. Application Data
(60) Provisional application No. 63/132,882, filed on Dec. 31, 2020.

(51) Int. Cl. A42B 3/22

(52) U.S. Cl. CPC A42B 3/221; A42B 3/225

(57) ABSTRACT

A safety helmet onto which a disposable face shield can be attached is provided. The safety helmet includes an outer shell and a bill extending from the outer shell. At least one integrally formed face shield attachment portion extends from a side of the safety helmet, for example a T-shaped tab. The disposable face shield has at least on slot cut into it which engages with the at least one face shield attachment portion to rotatably and removably attach the disposable face shield to the safety helmet. The bill forms a seal with the disposable face shield when the disposable face shield is in the lowered position.

### RELATED APPLICATION(S)

The present application claims priority to U.S. Provisional Patent Application No. 63/132,882, filed Dec. 31, 2020, and entitled “Safety Helmet with Disposable Sanitary Face Shield” (Attorney Docket No. 65272US01) which is hereby incorporated by reference in its entirety.

### BACKGROUND

The present disclosure generally relates to safety helmets having a disposable face shield, for example a sanitary face shield.

Some safety helmets, for example hard hats, or any other safety helmet, include face shields. Such existing helmets and face shields, however, often require complex mechanisms to attached the face shield to the safety helmet, which are both expensive to manufacture and to maintain. Additionally, these complex mechanisms attaching the face shield to the safety helmet can be prone to malfunction or breakage as they consist of multiple parts made from different materials. Traditional face shields may also be difficult to swap out for a new, and clean, face shield. This may again result from the complexity of the mechanism holding the face shield to the safety helmet or the construction of the face shield itself. It may also be cost prohibitive to frequently swap out traditional face shields for a new one for sanitary reasons. Therefore, traditional face shields are not well suitable for use as a disposable sanitation shield for safety helmets.

Consequently, it may be desirable to use a cost effective disposable sanitary face shield that is both easily attachable to, and removable from, a safety helmet to allow for frequent replacement.

### SUMMARY

A safety helmet is disclosed. The safety helmet may comprise: an outer shell; a bill extending from the outer shell; a face shield attachment portion integrally formed with and extending from a side of the safety helmet; and a disposable face shield. The disposable face shield may be rotatable between a raised position and a lowered position. The face shield attachment portion may be operable to rotatably and removably attach the disposable face shield to the safety helmet. The bill may form a seal with the disposable face shield when the disposable face shield is in the lowered position.

In some embodiments, the face shield attachment portion may comprise a T-shaped tab comprising an elongate outer portion and a circular inner portion. In other embodiments, the disposable face shield further may comprise a slot cut into the disposable face shield. The slot may comprise: a first aperture positioned at a first end of the slot; a second aperture positioned at a second end of the slot; and a slit positioned between the first aperture and the second aperture. The slot may be operable to receive the elongate outer portion of the T-shaped tab and the first aperture and the second aperture are operable to receive the circular inner portion of the T-shaped tab, such that the disposable face shield is pivotable about the circular inner portion when attached to the safety helmet.

In some embodiments, the disposable face shield may be attached to the safety helmet in a first configuration when the circular inner portion of the T-shaped tab is positioned in the first aperture of the slot. The disposable face shield may be attached to the safety helmet in a second configuration when the circular inner portion of the T-shaped tab is positioned in the second aperture of the slot. In such an embodiment, the first configuration may be vertically offset from the second configuration.

In some embodiments, the elongate outer portion of the T-shaped tab may be operable to push against the disposable face shield when the disposable face shield is attached to the safety helmet, such that friction between the elongate outer portion and the disposable face shield retains the disposable face shield in a position. In some embodiments.

In some embodiments, the face shield attachment portion may comprise a first T-shaped tab positioned on a first side of the safety helmet and a second T-shaped tab positioned on a second and opposing side of the safety helmet. In such embodiments, the circular inner portion of the first T-shaped tab and the circular inner portion of the second T-shaped tab may be concentric. In other embodiments, the disposable face shield may further comprise a first slot positioned adjacent a first end of the disposable face shield and a second slot positioned adjacent a second opposing end of the disposable face shield. In further embodiments, the disposable face shield may be constructed of a single sheet of PET, PETG, or PC.

A disposable face shield is disclosed. The disposable face shield may comprise: a flexible plastic sheet; a first slot cut into and positioned adjacent a first end of the disposable face shield; and a second slot cut into and positioned adjacent a second opposing end of the disposable face shield. The first slot and the second slot may each comprise a first aperture positioned at a first end, a second aperture positioned at a second end, and a slit positioned between the first aperture and the second aperture. The disposable face shield may be operable to be attached to a safety helmet by engaging the first slot with a first face shield attachment portion and engaging the second slot with a second face shield attachment portion.

In some embodiments, the flexible plastic sheet may be transparent. In other embodiments, the flexible plastic sheet may comprise an anti-fog coating. In further embodiments, the flexible plastic sheet may comprise a single sheet of PET, PETG, or PC.

In some embodiments, the first face shield attachment portion and the second face shield attachment portion may each comprise a T-shaped tab integrally formed with and extending respectively from a first side of the safety helmet and a second opposing side of the safety helmet. The T-shaped tab may comprise an elongate outer portion and a circular inner portion. The circular inner portion of the T-shaped tab of the first face shield attachment portion and the circular inner portion of the T-shaped tab of the second face shield attachment portion may be concentric.

In some embodiments, the first slot and the second slot of the disposable face shield may be operable to receive the elongate outer portion of the T-shaped tab. In such embodiments, the first aperture and second aperture may be operable to receive the circular inner portion of the T-shaped tab, such that the disposable face shield is pivotable about the circular inner portion when attached to the safety helmet.

In some embodiments, the flexible plastic sheet when attached to the safety helmet may create a spring load that pushes the flexible plastic sheet against the elongate outer portion of the T-shaped tab thereby creating friction between the elongate outer portion and the flexible plastic sheet and retaining the disposable face shield in a position.

In some embodiments, the disposable face shield may be attached to the safety helmet in a first configuration when the circular inner portion of the T-shaped tab of each the first face shield attachment portion and the second face shield attachment portion is positioned in the first aperture respectively of the first slot and the second slot. In such embodiments, the disposable face shield may be attached to the safety helmet in a second configuration when the circular inner portion of the T-shaped tab of each the first face shield attachment portion and the second face shield attachment portion is positioned in the second aperture respectively of the first slot and the second slot. The first configuration may be vertically offset from the second configuration.

A method for attaching a disposable face shield to a safety helmet is disclosed. The method may comprise: inserting a first T-shaped tab extending from a first side of the safety helmet into a first slot cut into and adjacent a first side of the disposable face shield; bending the disposable face shield around an outer surface of the safety helmet; and inserting a second T-shaped tab extending from a second opposing side of the safety helmet into a second slot cut into and adjacent a second opposing side of the disposable face shield.

In some embodiments the method may further comprise adjusting a height of the disposable face shield relative to the safety helmet by respectively positioning a circular inner portion of the first T-shaped tab and the second T-shaped tab in either a first aperture of the first slot and the second slot or a second aperture of the first slot and the second slot.

### BRIEF DESCRIPTION OF THE DRAWINGS

FIG. 1 is an exemplary front perspective view of a safety helmet with a disposable sanitary face shield, according to some aspects of the present disclosure.

FIG. 2 is an exemplary front elevation view of a safety helmet with a disposable sanitary face shield, according to some aspects of the present disclosure.

FIG. 3 is an exemplary rear elevation view of a safety helmet with a disposable sanitary face shield, according to some aspects of the present disclosure.

FIG. 4 is an exemplary right side elevation view of a safety helmet with a disposable sanitary face shield, according to some aspects of the present disclosure.

FIG. 5 is an exemplary left side elevation view of a safety helmet with a disposable sanitary face shield, according to some aspects of the present disclosure.

FIG. 6 is an exemplary top plan view of a safety helmet with disposable a sanitary face shield, according to some aspects of the present disclosure.

FIG. 7 is an exemplary bottom plan view of a safety helmet with disposable a sanitary face shield, according to some aspects of the present disclosure.

FIG. 8 is another exemplary perspective view of a safety helmet with a disposable sanitary face shield, showing that the face shield in a raised position, according to some aspects of the present disclosure.

FIG. 9 is an exemplary rear perspective view of a safety helmet with disposable a sanitary face shield, according to some aspects of the present disclosure.

FIG. 10 is an exemplary enlarged view of T-shaped tab and slot for connecting a disposable sanitary face shield to a helmet, according to some aspects of the present disclosure.

FIG. 11 is an exemplary partial cross sectional view of FIG. 10 of the T-shaped tab, showing how the disposable sanitary face shield may attach to the safety helmet, according to some aspects of the present disclosure.

FIG. 12 is an exemplary top plan view of a disposable face shield in an unattached configuration, according to some aspects of the present disclosure.

FIG. 13 is a flowchart of an exemplary method of attaching a disposable face shield to a safety helmet, according to some aspects of the present disclosure.

Although specific features of various embodiments may be shown in some drawings and not in others, this is for convenience only. Any feature of any drawing may be referenced and/or claimed in combination with any feature of any other drawing.

Unless otherwise indicated, the drawings provided herein are meant to illustrate features of embodiments of the disclosure. These features are believed to be applicable in a wide variety of systems comprising one or more embodiments of the disclosure. As such, the drawings are not meant to include all conventional features known by those of ordinary skill in the art to be required for the practice of the embodiments disclosed herein.

### DETAILED DESCRIPTION

The following detailed description illustrates embodiments of the disclosure by way of example and not by way of limitation. Specifically, embodiments of a safety helmet and attachable disposable sanitary face shield are described herein. Conventional safety helmets and face shields require complex attachment mechanisms and are too costly to frequently replace the face shield for sanitary reasons. As sanitation becomes ever more important, there is an increased need to frequently replace face shields on safety helmets. The disclosed embodiments of the disposable sanitary face shield and safety helmet with integral T-shaped tabs are both simple to use and simple to manufacture. The disclosed embodiments eliminate the complicated and heavy mechanism normally associated with a safety helmet mounted face shields. Thus, the simplicity and the disposable nature of the face shield make it ideal for sanitation use.

In some embodiments the safety helmet has a pair of T-shaped tabs extending from the sides of the safety helmet. These T-shaped tabs can be integral with the safety helmet (i.e., formed during the injection molding process) or a separate piece clipped onto the side of the safety helmet. In some embodiments the T-shaped tabs are sized to fit into and be inserted into a slot cut into each side of the face shield. As a result, the face shield can be easily attached and removed from the safety helmet by sliding the T-shaped tab into and out of the slots on the face shield. Additionally, in some embodiments, because the face shield is a flexible piece of plastic that is bent around the outer surface of the safety helmet when attached, the natural spring load of the face shield results in the face shield pushing against the T-shaped tabs when attached. Consequently, this contact between the face shield and T-shaped tabs results in friction, which is sufficient to hold the face shield in place when it is moved to and between the raised position and lowered position.

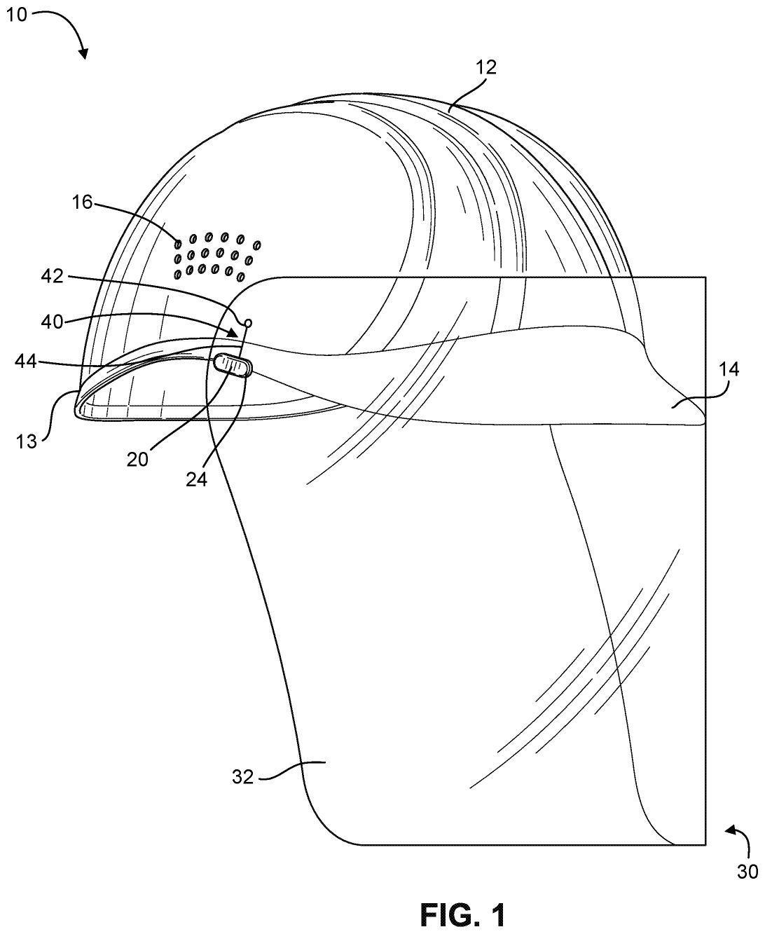

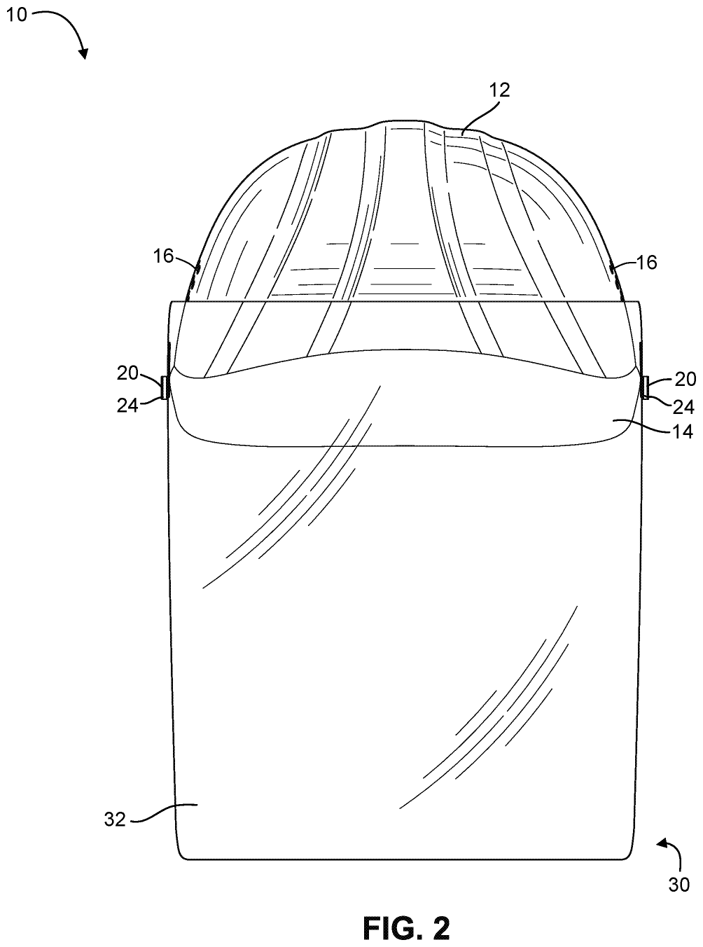

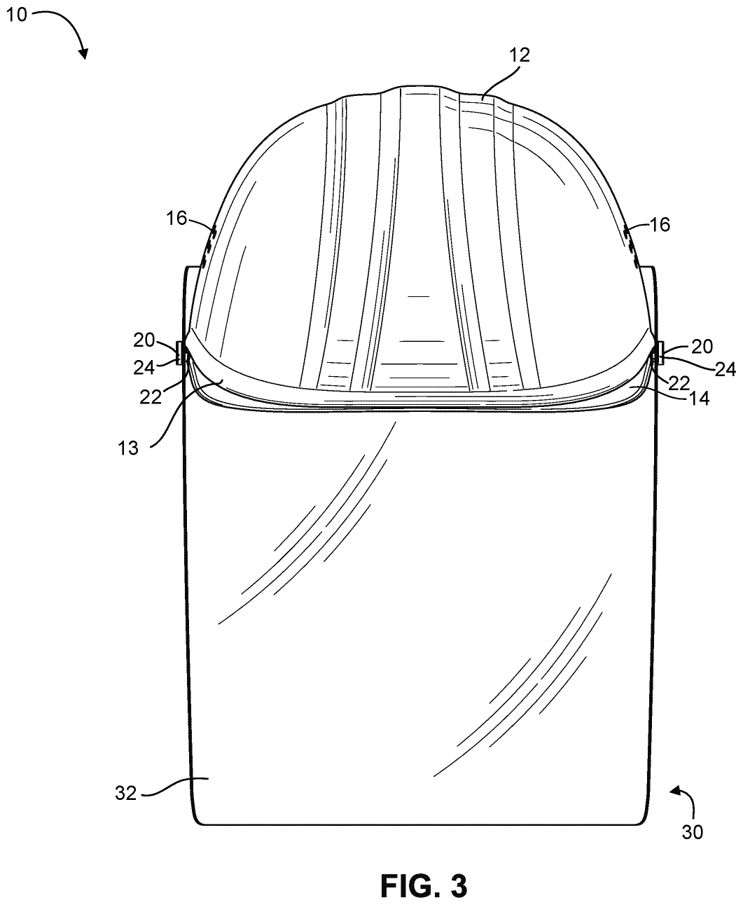

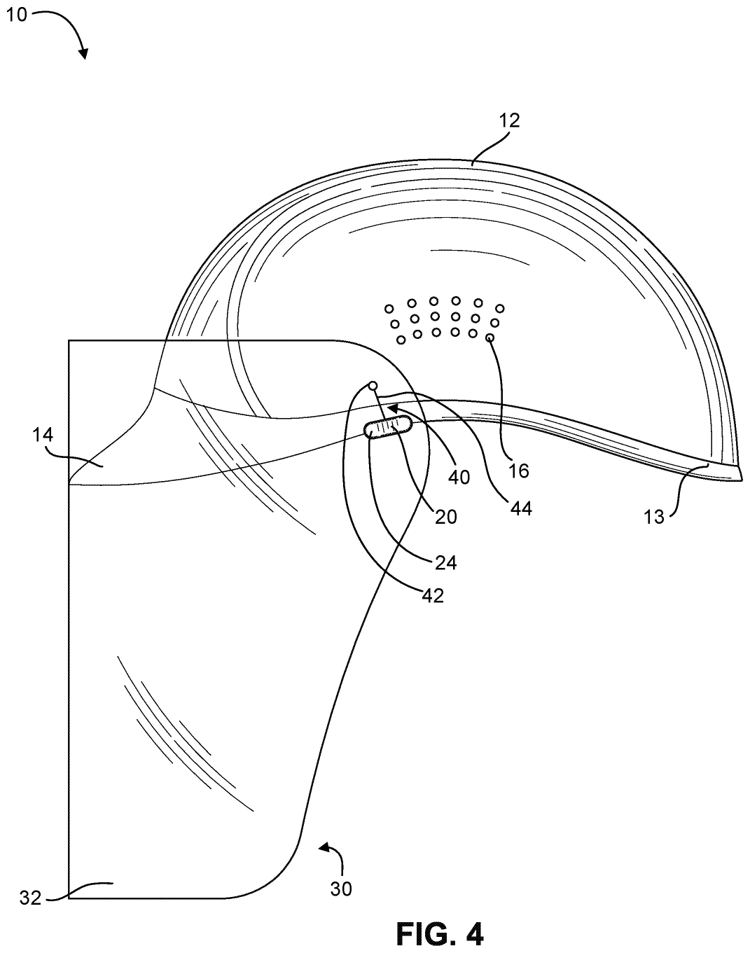

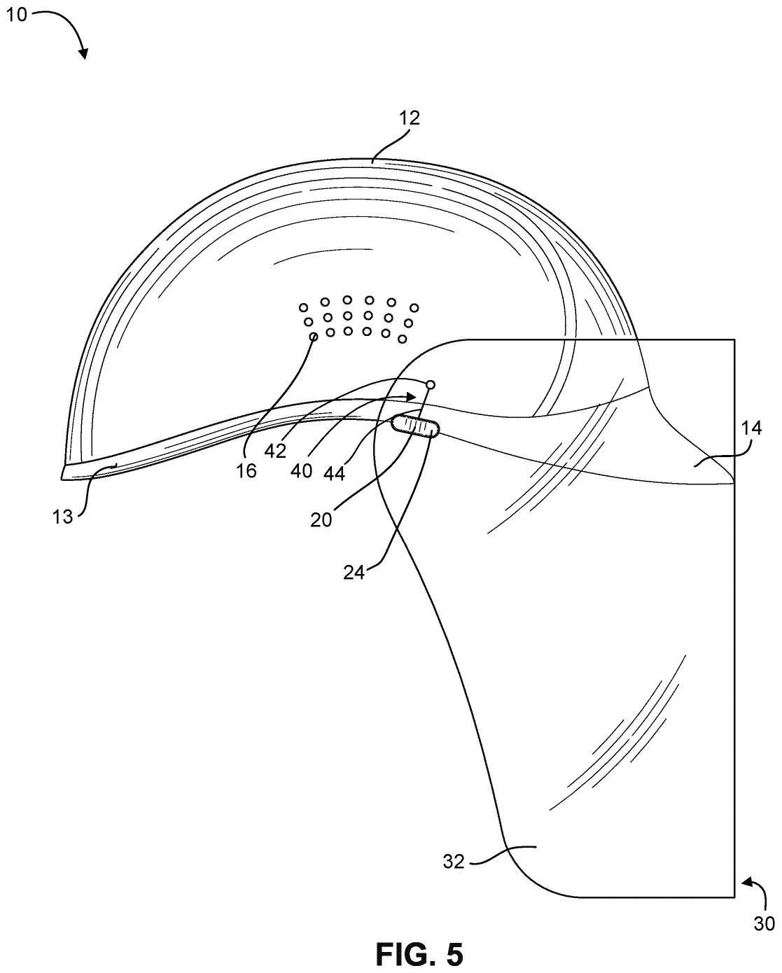

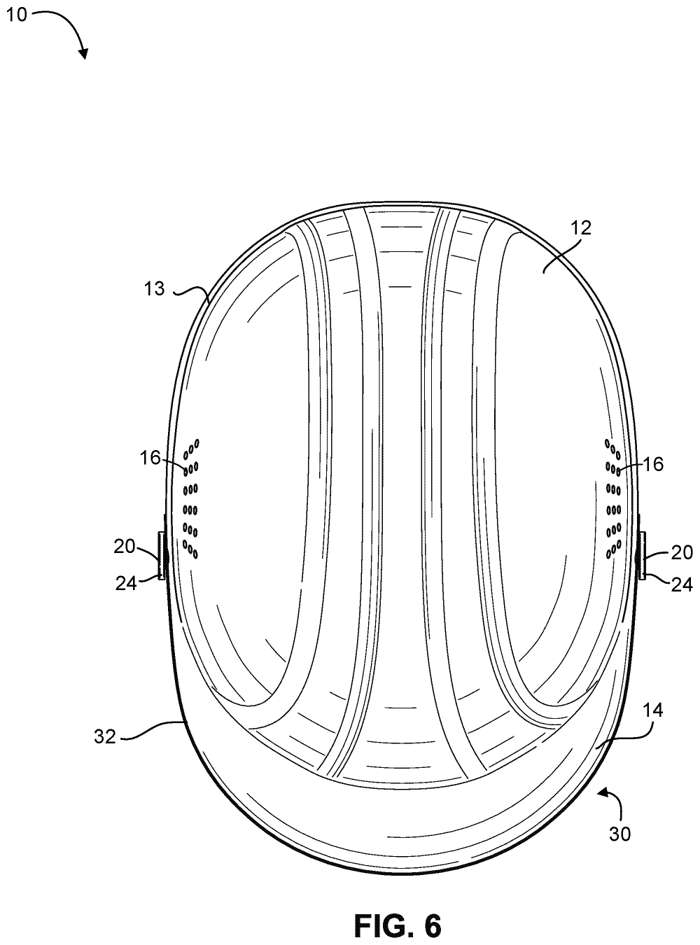

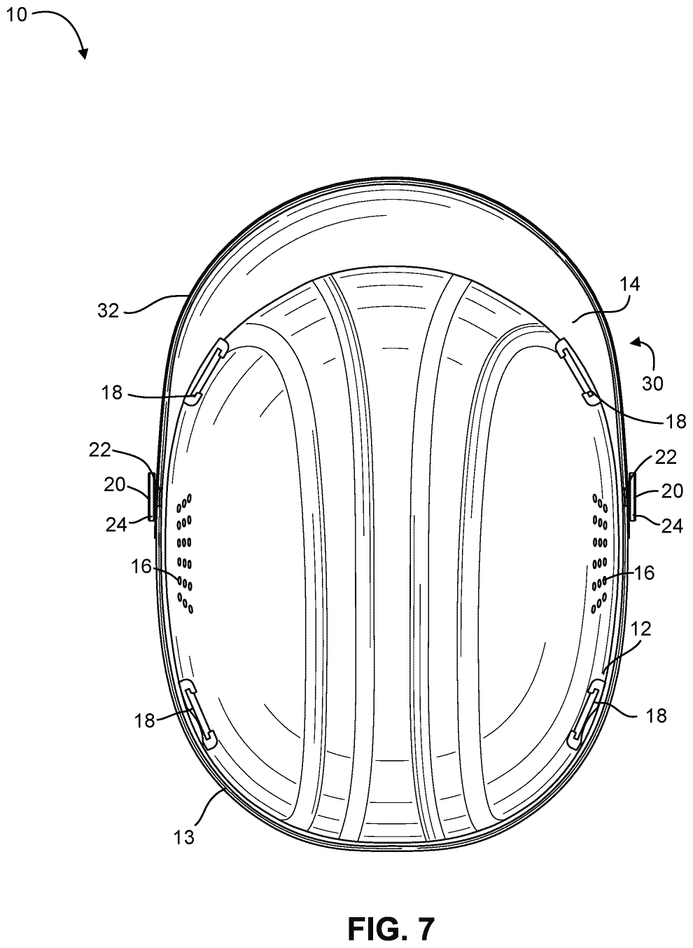

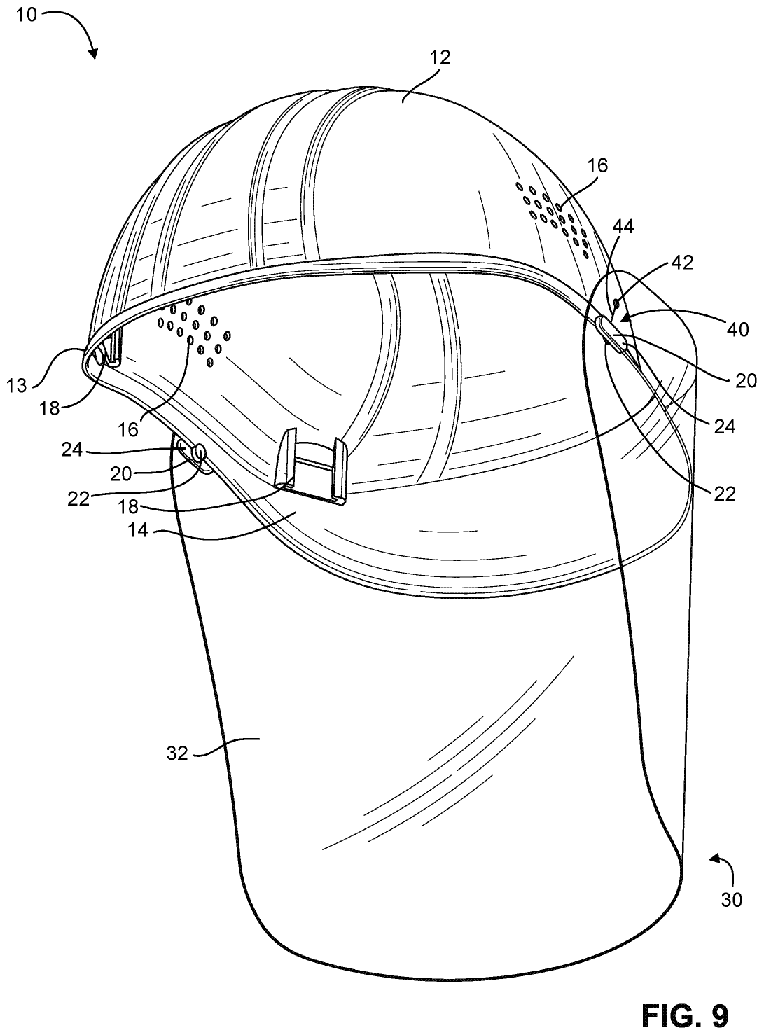

FIGS. 1-11 show an exemplary embodiment of a safety helmet 10 (e.g., a bump cap or hardhat) including a removable, and disposable, sanitary face shield 30. Specifically, FIGS. 1-11 illustrate a disposable sanitary face shield 30 that attaches to one or more face shield attachment portion(s) (e.g., a T-shaped tab 20) molded integrally to the safety helmet 10 or otherwise fastened to the safety helmet 10. In one embodiment, the safety helmet 10 includes for example an outer shell 12, a brim 13, a bill 14, one or more vent holes 16, one or more harness mounts 18, and two T-shaped tabs 20 concentrically aligned and positioned on opposite sides of the safety helmet 10. The outer shell 12 may be made from a single molded piece of high-density polyethylene (HDPE). The brim 13 may fully or partially surround the head opening of the safety helmet 10. The bill 14, may be integral with the outer shell 12 (i.e., part of the same single molded shell) or a separate piece clipped onto the outer shell 12. The harness mount(s) 18, shown for example in FIG. 9, may be the female portion of a release buckle to which a male portion of a release buckle attached to a harness or suspension straps (not shown) can be clipped. The harness mount(s) 18 may be an integrally molded component or a separate component fastened to the outer shell 12 of the safety helmet 10.

In one embodiment, T-shaped tab 20 extend outwards from the brim 13 of the safety helmet 10. The T-shaped tabs 20 may include a circular inner portion 22 and an elongate outer portion 24. The circular inner portion 22 may have a diameter not exceeding the narrowest portion of the elongate outer portion 24. In a preferred embodiment, the T-shaped tab(s) 20 may be integrally formed with the outer shell 12 of the safety helmet 10, such that the T-shaped tab(s) cannot be removed. In such a preferred embodiment, the safety helmet 10 is manufactured by injection molding the safety helmet 10 with the T-shaped tabs 20 integral to the outer shell 12 of the safety helmet 10. The present disclosure contemplates, however, alternative embodiments in which the T-shaped tabs 20 are be part of a separate piece that may be attached (for exampled clipped) to the side an existing safety helmet, thereby allowing attachment of a disposable sanitary face shield 30 to existing safety helmets. The disposable sanitary face shield 30 may be rotatably, and removably, attachable to the safety helmet 10 by snapping it over the T-shaped tab(s) 20. In such an embodiment, the T-shaped tab 20 serves as attachment point and becomes a swivel location for the face shield 30 to rotate into a lowered or raised position. Thus, once attached to T-shaped tabs 20 of the safety helmet 10, the face shield 30 can swivel or pivot about or around the T-shaped tab(s) 20 between a raised or a lowered position.

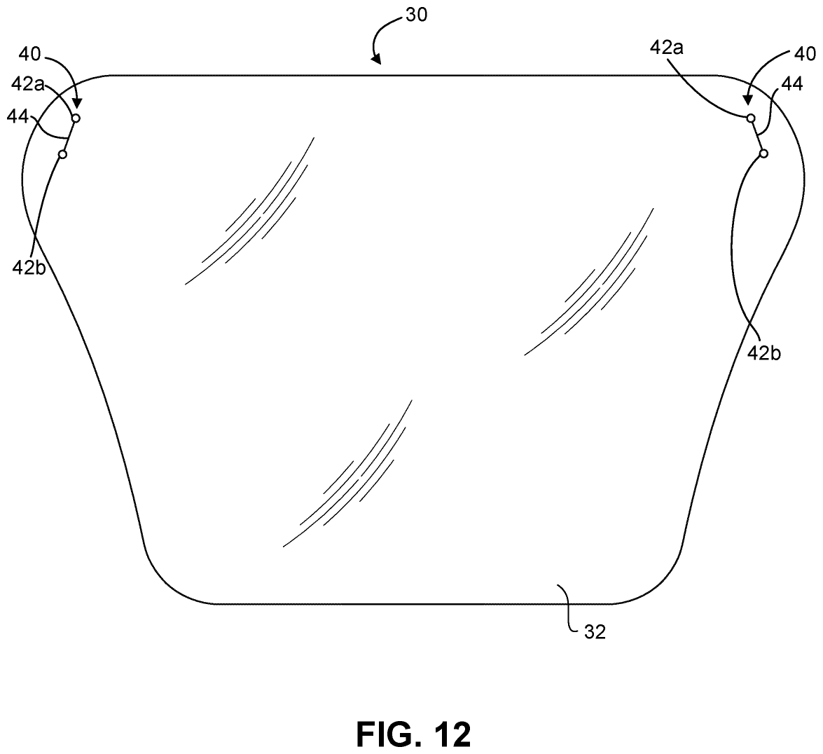

The disposable face shield 30, as shown in the example figures and specifically FIG. 12, may include generally a flexible plastic sheet 32, which has one or more slot(s) 40 cut into it. For example, in one embodiment the disposable face shield 30 has two slots 40 cut into upper and opposing sides. The disposable face shield 30 may be fully transparent or translucent. The disposable face shield 30 may also have a tint and or UV protection to protect against the sun. Additionally, the face shield 30 may include an anti-fog coating.

In on embodiment the disposable face shield 30 is a non-ANSI product, designed to be a disposable after use to ensure a maximum sanitary benefit to the user. For example, the disposable face shield 30 may be designed for use in any environment where a safety helmet 10 is worn and the wearer desires a barrier to their face for sanitation purposes. In such an embodiment, the disposable sanitary face shield 30 may not designed to provide impact protection as a traditional face shield apparatus would. As a result, the disposable sanitary face shield 30 may more cost effective to manufacture. In another embodiment, the face shield 30 may be a certified ANSI product and also provide some impact resistance.

The disposable face shield 30 may be constructed from a single sheet of stock polyethylene terephthalate (“PET”), polyethylene terephthalate glycol (“PETG”), or polycarbonate (“PC”). Specifically, the disposable face shield 30 may be die cut (using a steel rule die or similar) from a sheet (or roll) of PET, PETG, or PC. For example, 0.020 inch thick stock PET or may be used to form the disposable face shield 30, but thicker or thinner material could also be used. In one embodiment, a Protective film may be left on the one or more sides of the stock PET/PETG while cutting and handling to protect the disposable face shield 30 from scratches prior to use. The slot(s) 40 may be cut into the face shield 30 using the same or different die.

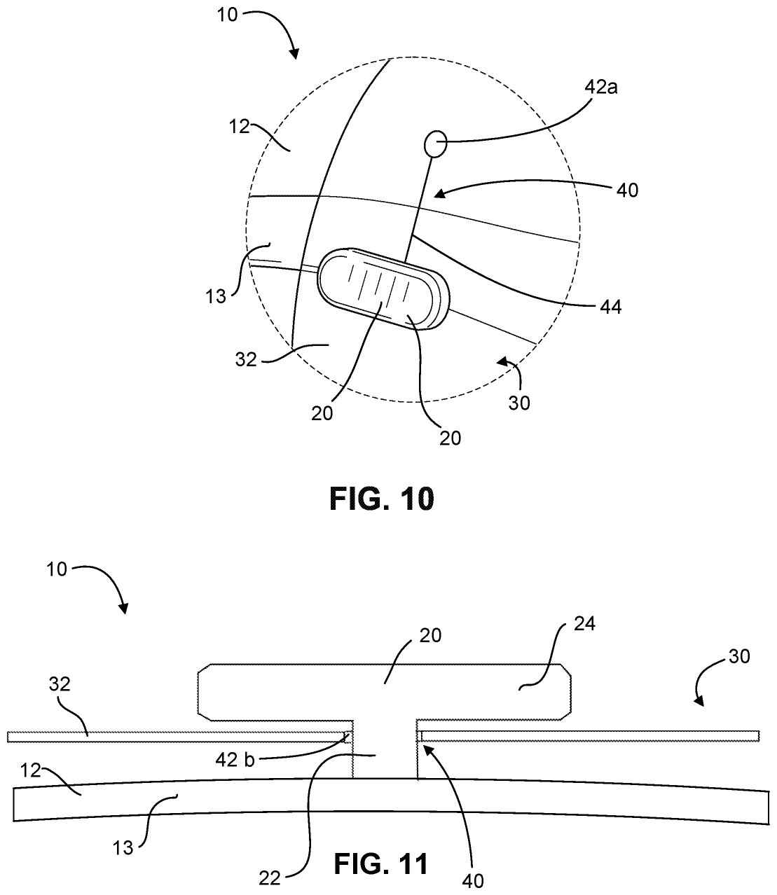

In one embodiment, the slot(s) 40 includes of one or more apertures 42. For example as shown in FIGS. 10-12, the slot(s) 40 may have a first aperture 42a positioned at a first end of the slot 40 and a second aperture 42b positioned at a second end of the slot 40. The slot 40 may also include a slit 44 positioned between the first aperture 42a and second aperture 42b. The present disclosure also contemplates other embodiments including for example a single aperture 42 positioned at an end of the slit 44. The slot(s) 40, may be operable to engage with the T-shaped tab 20 to serve as an attachment and pivot point for the disposable face shield 30. Specifically, the aperture(s) 42 may be sized to help provide the sufficient clearance to insert the elongate outer portion 24 of the T-shaped slots 20. Additionally, the aperture(s) may contribute to the flexibility of the face shield material (and slot(s) 40) such that the disposable face shield 30 can be slid over and engaged with the T-shaped tabs 20.

FIG. 11 shows an exemplary close-up view of the T-shaped tab 20 positioned in a slot 40. FIG. 12 shows an exemplary cross section of FIG. 11. In such an embodiment, aperture(s) 42 (i.e., first aperture 42a and second aperture 42b) have a diameter the same or slightly smaller than the diameter of the circular inner portion 22 of the T-shaped tab 20. Therefore, when the disposable face shield 30 is attached to the safety helmet 10, the circular inner portion 22 of the T-shaped tabs 20 will be positioned in the aperture and the face shield 30 can rotate about circular inner portions 22 of the T-shaped tabs 20. In the embodiment having two T-shaped tabs 20, the circular inner portions 22 are concentrically aligned creating an axis of rotation.

In one embodiment, T-shaped tabs 20 may also be operable to retain the face shield 30 onto the safety helmet 10. For example, because the elongate outer portions 24 of the T-shaped tabs 20 are significantly wider than the circular inner portion 22 (and apertures 42), the disposable face shield 30 cannot be removed from the safety helmet without first rotating the face shield 30 into a specific position aligning the slot 40 with the elongate outer portion 24. This alignment position may for example be mid-way through the arc between a completely raised position and a lowered position to avoid accidental removal of the face shield 30 in the raised and lowered positions.

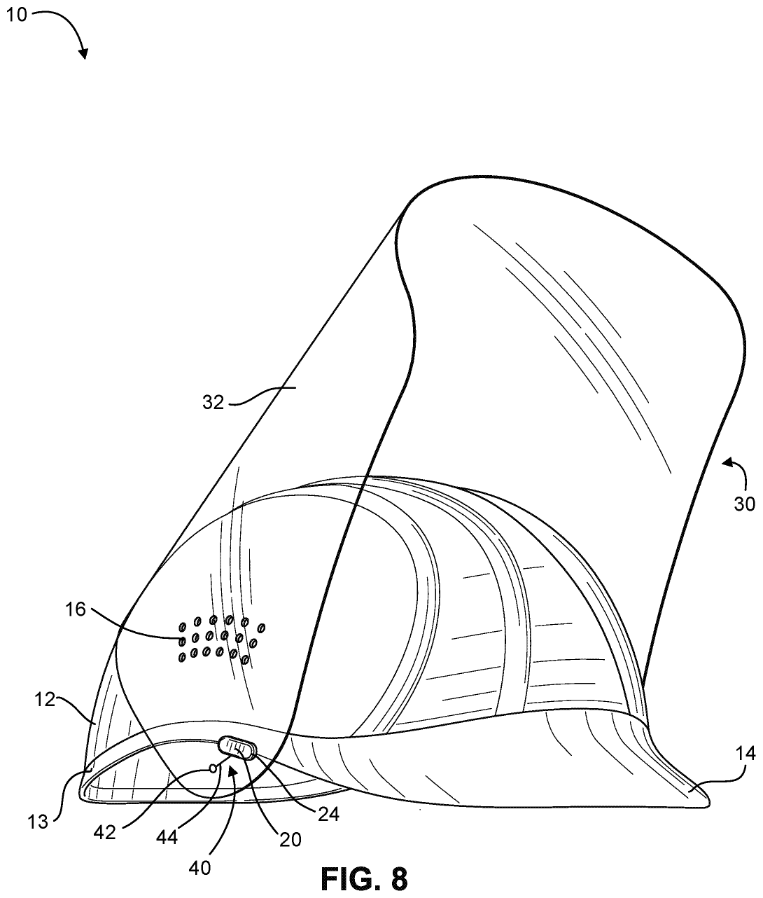

In one embodiment, the T-shaped tabs 20 may contribute to holding the face shield 30 in position in the raised, the lowered, or any intermediate position. For example, the because the face shield 30 in its unassembled state is a flexible but flat piece of material, when bent into its assembled state it has a natural spring load. This spring load may contribute to pushing the face shield 30 against T-shaped tab 20, and specifically against the elongate outer portion 24, such that friction between the elongate portion 24 and the face shield 30 helps to hold the face shield 30 in place. Additionally, as shown for example in FIG. 8, when the face shield 30 is rotated into the raised position (i.e., pushed back against the upper surface of the safety helmet 10) friction between the T-shaped tabs 20 and/or upper surface of the safety helmet 10 with the face shield 30 may retain the face shield 30 against the safety helmet 10.

In one embodiment, the T-shaped tabs 20 may be positioned on the brim 13 such that the face shield 30, when attached, creates an effective seal between the face shield 30 and safety helmet 10. In such an embodiment, the linear length between the two slots 40 of the face shield 30 may be approximately equal the arc length along the section of the brim 13 and/or bill 14 between the two T-shaped tabs 20. Thus, when the face shield 30 is in the lowered position, as shown for example in FIGS. 1-5 and 9, it rests (and is retained by) the brim 13 and/or bill 14 of the safety helmet 10 effectively sealing the area below the brim 13 and/or bill 14 of the safety helmet 10.

In one embodiment, the disposable face shield 30 may be height adjustable relative to the safety helmet. For example, the disposable face shield 30 may be attached to the safety helmet 10 in a first configuration (e.g., higher configuration) when the circular inner portion 22 of the T-shaped tab 20 is positioned in the first aperture 42a of the slot 40. Conversely, the disposable face shield 30 may be attached to the safety helmet 10 in a second configuration (e.g., lower configuration) when the circular inner portion 22 of the T-shaped tab 20 is positioned in the second aperture 42b of the slot 40. Thus, if the disposable face shield 30 is attached in first configuration, it is vertically offset and positioned higher (relative to the brim 13 and/or bill 14 of the safety helmet 10) than if the disposable face shield 30 is attached in the second configuration, or vice-versa.

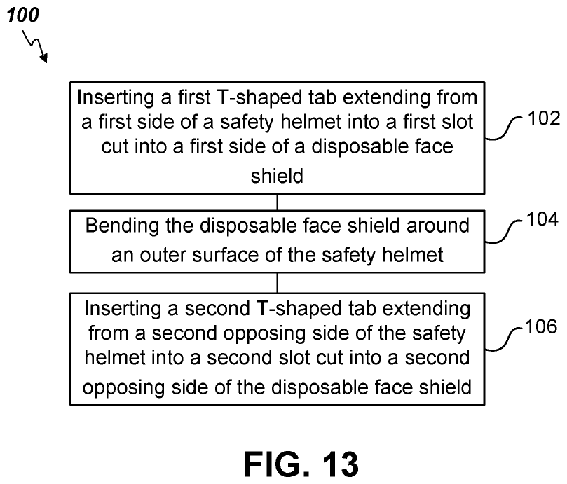

FIG. 13 provides an exemplary method 100 for attaching a disposable face shield 30 to a safety helmet 10. In block 102, a first T-shaped tab 20 extending from a first side of the safety helmet 10 is inserted into a first slot cut into a first side of the disposable face shield. In block 104, the disposable face shield 30 is bent around an outer surface of the safety helmet 10. In block 106, a second T-shaped tab 20 extending from a second opposing side of the safety helmet 10 is inserted into a second slot cut into a second opposing side of the disposable face shield. It is to be understood that the present disclosure is not limited the specific order of blocks 102 to 106—other orders are possible. The Method steps may also be performed in parallel.

The exemplary method may also include steps for adjusting a height of the disposable face shield 30 relative to the safety helmet 10 by respectively positioning a circular inner portion of the first T-shaped tab 20 and the second T-shaped tab 20 in either a first aperture 42a of the first and second slots 40 or a second aperture 42b of the first and second slots.

While the disclosure has been described with respect to the figures, it will be appreciated that many modifications and changes may be made by those skilled in the art without departing from the spirit of the disclosure. Any variation and derivation from the above description and figures are included in the scope of the present disclosure as defined by the claims.

What is claimed is:

1. A safety helmet, comprising:
an outer shell;
a bill extending from the outer shell;
a face shield attachment portion integrally formed with and extending from a side of the safety helmet; and
a disposable face shield, rotatable between a raised position and a lowered position,
wherein the face shield attachment portion is operable to rotatably and removably attach the disposable face shield to the safety helmet and the bill forms a seal with the disposable face shield when the disposable face shield is in the lowered position.

2. The safety helmet of claim 1, wherein the face shield attachment portion comprises a T-shaped tab comprising an elongate outer portion and a circular inner portion.

3. The safety helmet of claim 2, wherein the disposable face shield further comprises a slot cut into the disposable face shield, the slot comprising:
a first aperture positioned at a first end of the slot;
a second aperture positioned at a second end of the slot; and
a slit positioned between the first aperture and the second aperture.

4. The safety helmet of claim 3, wherein:
the slot is operable to receive the elongate outer portion of the T-shaped tab; and
the first aperture and the second aperture are operable to receive the circular inner portion of the T-shaped tab, such that the disposable face shield is pivotable about the circular inner portion when attached to the safety helmet.

5. The safety helmet of claim 4, wherein:
the disposable face shield is attached to the safety helmet in a first configuration when the circular inner portion of the T-shaped tab is positioned in the first aperture of the slot;
the disposable face shield is attached to the safety helmet in a second configuration when the circular inner portion of the T-shaped tab is positioned in the second aperture of the slot; and
the first configuration is vertically offset from the second configuration.

6. The safety helmet of claim 4, wherein the elongate outer portion of the T-shaped tab is operable to push against the disposable face shield when the disposable face shield is attached to the safety helmet, such that friction between the elongate outer portion and the disposable face shield retains the disposable face shield in a position.

7. The safety helmet of claim 6, wherein the face shield attachment portion comprises a first T-shaped tab positioned on a first side of the safety helmet and a second T-shaped tab positioned on a second and opposing side of the safety helmet.

8. The safety helmet of claim 7, wherein the circular inner portion of the first T-shaped tab and the circular inner portion of the second T-shaped tab are concentric.

9. The safety helmet of claim 8, wherein the disposable face shield further comprises a first slot positioned adjacent a first end of the disposable face shield and a second slot positioned adjacent a second opposing end of the disposable face shield.

10. The safety helmet of claim 1, wherein the disposable face shield is constructed of a single sheet of PET, PETG, or PC.

11. A disposable face shield, comprising:
a flexible plastic sheet;
a first slot cut into and positioned adjacent a first end of the disposable face shield; and
a second slot cut into and positioned adjacent a second opposing end of the disposable face shield, wherein:
the first slot and the second slot each comprise a first aperture positioned at a first end, a second aperture positioned at a second end, and a slit positioned between the first aperture and the second aperture, and
the disposable face shield is operable to be attached to a safety helmet by engaging the first slot with a first face shield attachment portion and engaging the second slot with a second face shield attachment portion.

12. The disposable face shield of claim 11, wherein the flexible plastic sheet is transparent.

13. The disposable face shield of claim 11, wherein the flexible plastic sheet comprises an anti-fog coating.

14. The disposable face shield of claim 11, wherein the flexible plastic sheet comprises a single sheet of PET, PETG, or PC.

15. The disposable face shield of claim 11, wherein:
the first face shield attachment portion and the second face shield attachment portion each comprise a T-shaped tab integrally formed with and extending respectively from a first side of the safety helmet and a second opposing side of the safety helmet;
the T-shaped tab comprises an elongate outer portion and a circular inner portion; and
the circular inner portion of the T-shaped tab of the first face shield attachment portion and the circular inner portion of the T-shaped tab of the second face shield attachment portion are concentric.

16. The disposable face shield of claim 15, wherein:
the first slot and the second slot of the disposable face shield are operable to receive the elongate outer portion of the T-shaped tab; and
the first aperture and the second aperture are operable to receive the circular inner portion of the T-shaped tab, such that the disposable face shield is pivotable about the circular inner portion when attached to the safety helmet.

17. The disposable face shield of claim 16, wherein the flexible plastic sheet when attached to the safety helmet creates a spring load that pushes the flexible plastic sheet against the elongate outer portion of the T-shaped tab thereby creating friction between the elongate outer portion and the flexible plastic sheet and retaining the disposable face shield in a position.

18. The disposable face shield of claim 16, wherein:
the disposable face shield is attached to the safety helmet in a first configuration when the circular inner portion of the T-shaped tab of each the first face shield attachment portion and the second face shield attachment portion is positioned in the first aperture respectively of the first slot and the second slot;
the disposable face shield is attached to the safety helmet in a second configuration when the circular inner portion of the T-shaped tab of each the first face shield attachment portion and the second face shield attachment portion is positioned in the second aperture respectively of the first slot and the second slot; and
the first configuration is vertically offset from the second configuration.

19. A method for attaching a disposable face shield to a safety helmet, the method comprising:
inserting a first T-shaped tab extending from a first side of the safety helmet into a first slot cut into a first side of the disposable face shield;
bending the disposable face shield around an outer surface of the safety helmet; and
inserting a second T-shaped tab extending from a second opposing side of the safety helmet into a second slot cut into a second opposing side of the disposable face shield.

20. The method of claim 19, further comprising adjusting a height of the disposable face shield relative to the safety helmet by respectively positioning a circular inner portion of the first T-shaped tab and the second T-shaped tab in either a first aperture of the first slot and the second slot or a second aperture of the first slot and the second slot.
